(19) United States
(12) Patent Application Publication
(10) Pub. No.: US 2024/0050647 A1
(43) Pub. Date: Feb. 15, 2024

(54) DUAL CHAMBER BLOOD HAND PUMP

(71) Applicant: CAREFUSION 303, INC., San Diego, CA (US)

(72) Inventors: SHEETHAL RAO, Bangalore (IN); Karthik MR, Bangalore (IN)

(21) Appl. No.: 17/888,197

(22) Filed: Aug. 15, 2022

(51) Int. Cl. A61M 5/142; A61M 5/14; A61M 5/168

(52) U.S. Cl. CPC A61M 5/1424; A61M 5/1414; A61M 5/16813; A61M 5/1422

(57) ABSTRACT

A dual chamber single-hand pump is described that includes a pump chamber with four fluid pathways. Two pathways are configured to conduct fluid to the chamber and to stop flow therethrough from the chamber, and two pathways are configured to conduct fluid from the chamber and to stop flow therethrough to the chamber. A piston positioned and moveable within the chamber separates two of the pathways from the other two pathways within the chamber. The pump can be held in a single hand by having finger grips for stabilizing the pump and a palm grip for advancing the piston. A return spring can return the piston to the original position when the palm is relaxed to allow expansion of the spring.

### BACKGROUND

Therapeutic treatments can include the preparation of equipment and infusion of a medical fluid (e.g., blood, plasma, saline). This is prepared for and communicated to patients using an IV catheter that is connected though an arrangement of flexible tubing and fittings, commonly referred to as an “IV set.” The equipment often includes connection to a source of fluid, for example, an IV blood bag. During operation or use, medical fluid may be required quickly at greatly increased flow rates, as shorter times to perform blood transfusions have been associated with decreased death risk in trauma patients. Typical IV sets use a cylindrical hand pump that is squeezed by hand to rapidly increase fluid flow rate, resulting in muscle fatigue.

### SUMMARY

For these reasons, it is desirable to provide an IV set hand pump that is constructed to operate without extensive effort or time and with a single hand by the user. This can help reduce hand muscle fatigue and complexity of use.

This subject technology provides an IV set designed for delivery of fluids at high flow rates, particularly addressing the delivery of large volumes of blood in trauma situations. Described herein are single-hand pumps for use with an intravenous delivery system. The pumps can include a pump chamber comprising a plurality of ports that provide fluid pathways through a wall of the chamber housing; and a plunger positioned within the chamber and configured to be reciprocally axially moveable in the chamber, the plunger separating the chamber into a first chamber and a second chamber. Some embodiments provide that the first chamber is in fluid communication with a first plurality of ports, the second chamber is in fluid communication with a second plurality of ports, the first plurality of ports being fluidly isolated through the chamber from the second plurality of ports by the plunger. In some embodiments, when the plunger is axially advanced toward the first plurality of ports, fluid is expelled from the chamber through a first of the first plurality of ports and restricted from being expelled through a second of the first plurality of ports, and fluid is conducted into the chamber through a first of the second plurality of ports and restricted from being conduct into the chamber through a second of the second plurality of ports. Some embodiments provide that when the plunger is axially advanced toward the second plurality of ports, fluid is expelled from the chamber through one of the second plurality of ports and restricted from being expelled through another of the second plurality of ports, and fluid is conducted into the chamber through a one of the first plurality of ports and restricted from being conduct into the chamber through another of the second plurality of ports.

Described herein are embodiment that further include a return spring that is compressed when the plunger is advanced in a first direction and expands when the plunger is advanced in a second direction. Some embodiments include a handle that is configured to be pressed by a palm of a user, and some include a plurality of finger grips that are configured to be engaged by a user's fingers. In some embodiments, the single-hand pump is configured to be actuated by a single hand of a user as the plunger advances toward the first plurality of ports and as the plunger advances toward the second plurality of ports.

In some of the embodiments described herein, the first plurality of ports comprises at least two one-way valves, and in some embodiments, the at least two one-way valves comprise ball valves. In some embodiments, the second plurality of ports comprises at least two one-way valves, and in some embodiments, the at least two one-way valves comprise ball valves.

Some embodiments of the subject technology described herein include a single-hand pump for use with an intravenous delivery system that has a pump chamber comprising a first fluid pathway, a second fluid pathway, a third fluid pathway, and a fourth fluid pathway, the first and third pathways configured to conduct fluid to the chamber and to stop flow therethrough from the chamber, the second and fourth pathways configured to conduct fluid from the chamber and to stop flow therethrough to the chamber. Some pumps described herein include a piston positioned within the chamber, the piston being moveable between first and second positions and fluidly separating the first and second fluid pathways from the third and fourth fluid pathways within the chamber, wherein when the piston is moving toward the first position, fluid is conducted through the second and third fluid pathways and stopped through the first and fourth fluid pathways, and when the piston is moving toward the second position, fluid is conducted through the first and fourth fluid pathways and stopped through the second and third fluid pathways.

Some embodiments further include first, second, third, and fourth ports fluidly coupled to the chamber providing respective flow along the first, second, third, and fourth fluid pathways. Some embodiments also include a plurality of one-way valves in each of the ports, the valves configured to conduct or stop the flow during movement of the piston. In certain embodiments, the plurality of one-way valves comprises a ball valve.

Some of the described embodiments include a plurality of finger grips that are configured to be engaged by a user's fingers, wherein the single-hand pump is configured to be actuated by a single hand of a user as the plunger moves between the first and second positions. In some embodiments, a return spring is compressed as the piston moves in one direction and expands as the piston moves in another direction, and the piston is configured to move in the one direction by a user's action, and the piston is configured to move in another direction by expansion of the return spring.

Methods of using a dual chamber pump are also described herein. The methods can be for treating a patient or for preparing a set of lines for treatment, also referred to as priming the set. Methods of conducting fluid through an intravenous delivery system using a single-hand pump can include providing a pump chamber comprising a first fluid pathway, a second fluid pathway, a third fluid pathway, and a fourth fluid pathway, the first and third pathways configured to conduct fluid to the chamber and to stop flow therethrough from the chamber, the second and fourth pathways configured to conduct fluid from the chamber and to stop flow therethrough to the chamber. Some methods include providing a piston positioned within the chamber, the piston fluidly separating the first and second fluid pathways from the third and fourth fluid pathways within the chamber. Some methods also include the step of moving the piston between first and second positions, and wherein when the piston is moving toward the first position, fluid is conducted through the second and third fluid pathways and stopped through the first and fourth fluid pathways, and when the piston is moving toward the second position, fluid is conducted through the first and fourth fluid pathways and stopped through the second and third fluid pathways.

Some methods may also include providing first, second, third, and fourth ports fluidly coupled to the chamber. In some instances, some methods further include conducting and stopping fluid flow along the first, second, third, and fourth pathways via one-way valves.

According to various aspects of the subject technology, an infusion system for intravenous delivery of a fluid from a fluid container, comprises a malleable fluid container configured to store a fluid and deliver the fluid via a connected infusion tubing; a fluid container pressure sleeve configured to wrap around, connect and form to the malleable fluid container such that, when the fluid container pressure sleeve is inflated with a gas, the fluid container pressure sleeve applies an inward directional pressure to the malleable fluid container from an exterior of the malleable fluid container; a pump configured to provide the gas to the fluid container pressure sleeve; and a pressure measuring device configured to measure a pressure associated with the malleable fluid container by the fluid container pressure sleeve.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure. Like reference numerals refer to corresponding parts throughout the figures and description.

FIG. 1 depicts a schematic view of a patient connected to an intravenous system incorporating aspects of the disclosure.

FIG. 2 depicts a schematic view of an intravenous system incorporating aspects of the disclosure.

FIG. 3 depicts a perspective view of a dual chamber hand pump in accordance with aspects of the disclosure.

FIG. 4 depicts a perspective view of a user's hand holding a dual chamber hand pump in accordance with aspects of the disclosure.

FIG. 5 depicts a cross-sectional view of a dual chamber hand pump in accordance with aspects of the disclosure.

FIG. 6 depicts an exploded partial cross-sectional view of a dual chamber hand pump in accordance with aspects of the disclosure.

FIG. 7 depicts a cross-sectional view of a position of a dual chamber hand pump in accordance with aspects of the disclosure for pumping in one direction.

FIG. 8 depicts a cross-sectional view of a position of a dual chamber hand pump in accordance with aspects of the disclosure for changing directions of a pumping action.

FIG. 9 depicts a cross-sectional view of a position of a dual chamber hand pump in accordance with aspects of the disclosure for pumping in a direction different than shown in FIG. 7.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, any dimensions provided are in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

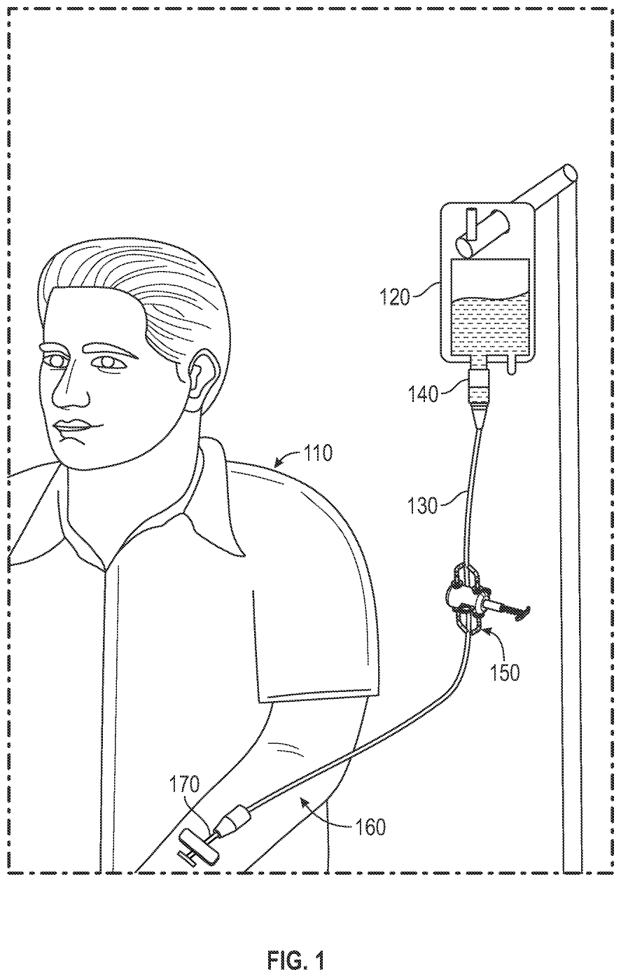

FIG. 1 depicts a schematic view of a patient 110 connected to an intravenous system incorporating aspects of the disclosure. Intravenous fluids, such as a blood infusate, are often contained in malleable (e.g., flexible) bags, commonly referred to as intravenous or “IV” bags. These bags, or fluid source 120, can be equipped with multiple septums or other fluid connections that permit connection of the bag to a tube that feeds the fluid to the patient 110. The bags are generally floppy, although the fluid sources 120 need not be floppy bags, and the bags may be subject to puncture if they come into contact with sharp items. Alternate containers, which are more prevalent in some countries, include glass bottles and soft plastic bottles.

Administration of these IV fluids, regardless of the container, requires that the fluid source 120 be suspended at some height, typically 0.5-1.0 meter, above the patient 110 or an infusion pump. This container is then connected by a flexible tube to either the patient 110 directly or to the infusion pump. Mounting the fluid source 120 above the delivery point generates a positive pressure due to gravity at the connection of the infusion tube to the patient 110 or pump. One embodiment of such a mounting is illustrated in FIG. 1, wherein an IV bag is mounted in an elevated position. An input line 130 is connected to the fluid source 120 via a drip chamber 140. Fluid flow is provided to the input line 130 through the drip chamber 140 from the fluid source 120.

Flow may be achieved by either gravity-pressure or positive-pressure. Gravity-pressure based flow control systems rely on the force of gravity for fluid flow. Such systems, generally referred to as gravity sets, may include an “IV controller” which interfaces with the IV tube. An IV controller may be a device that automatically controls the flow rate of fluid through the IV tube by use of a pinching device that pinches the tube at different degrees to control the flow of fluid therethrough. In some instances, an IV controller may be a manually manipulated device that a caregiver uses and adjusts to pinch the tubing. In some instances, an IV controller may also be responsive to a control signal generated by, for example, a flow sensor attached to the drip chamber. Advantages of gravity sets include their relative simplicity and low cost. The pinching device comprises a relatively simple mechanical device under electrical control. IV controllers, however, are limited to gravity pressure, dependent upon the “head height” or “head pressure” of the administration fluid, which can be under 1 psi.

In certain situations, the amount of pressure provided by a gravity-pressure based flow control device may be insufficient. In other situations, greater accuracy and precision of flow rates are required. In such situations, a positive pressure flow control device is necessary.

A separate infusion pump (not depicted) may be used to infuse the fluid with a greater flow rate than that dependent upon gravity. Some infusion pumps act as flow control devices to act on the respective tube or fluid conduit of the fluid administration set to move the fluid from the fluid container through the conduit to the patient 110 at a desired rate. However, in some circumstances, it may be required to use a gravity set without a separate infusion pump such as, for example, when a separate infusion pump is not available or accessible. Yet, in some instances, it may be desirable to provide fluid flow with gravity sets that provide a greater flow than that resulting from the positive pressure due to gravity.

Some instances where it may be desirable to increase fluid flow are, for example, when delivering large amounts of blood over a small period of time, such as in trauma situations. In such instances, a separate infusion pump may not be available, and it may be desirable to use an in-line hand pump, or in some cases pressure sleeves around the blood IV bag that are also manually pumped operated, to provide blood flow to the patient at a great flow than that achievable through a standard gravity set. With further reference to FIG. 1, embodiments in which a hand pump 150 is provided in line with a gravity set. As depicted, the hand pump 150 can be connected to the input line 130 on one end and an output line 160 on another end. The output line 160 can extend from the hand pump 150 to an IV injection set 170 that is configured to communicate fluid to the patient's 110 vasculature.

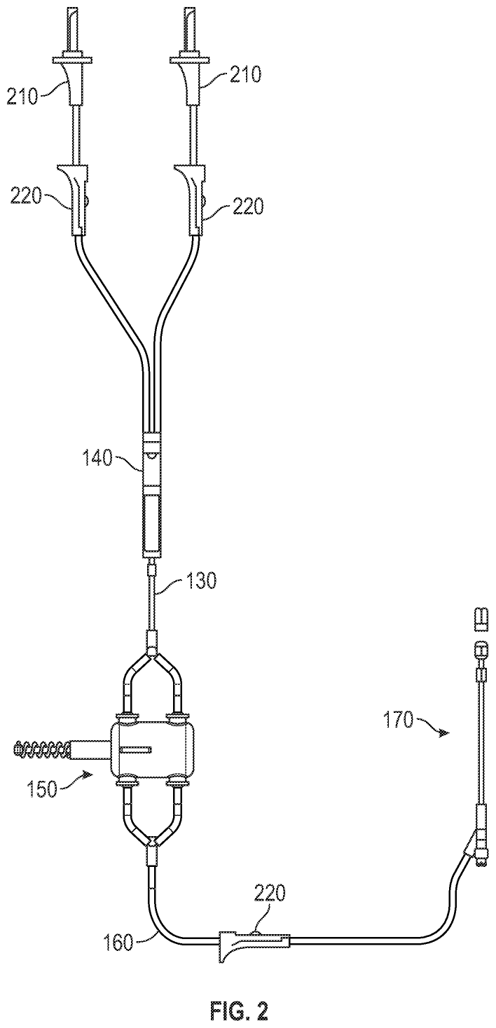

Medical fluid administration sets, including gravity sets, may have more parts than are shown in FIG. 1. For example, as illustrated in FIG. 2, IV sets may be formed from any combination of infusion components and tubing. Typically, the infusion components and tubing are disposable products that are used once and then discarded. The infusion components and tubing may be formed from any suitable material (e.g., plastic, silicone, rubber, PVC), many or all of which are clear or translucent so that the fluid flow or levels inside can be seen.

As shown in FIG. 2, IV sets may include one or more IV bag needles 210 and one or more roller clamps 220 connected by tubing. The IV set may also include a Y-site or a drip chamber 140 that combines the tubing from the one or more IV bag needles 210. The drip chamber 140 may be connected to the input line 130, the hand pump 150, and the output line 160. Flow through the output line may be controlled by another roller clamps 220. And the output line 160 may be connected to an IV injection set 170 that is configured for delivering fluid to the patient 110 (shown in FIG. 1).

In use, IV set is connected to a fluid source 120 (e.g., a blood bag) via the drip chamber 140, the input line 130, the hand pump 150, and the output line 160. The output line 160 is connected to a catheter that is placed into a vein of a patient. Thus, fluid flows from the fluid source 120, through the drip chamber 140 to the hand pump 150 and through the remainder of the IV set and out of the injection set 170. As the hand pump 150 is actuated or squeezed, the volume of fluid contained within a hand pump chamber 310 is forced out of the hand pump 150 through the output line 160 and downstream through the injection set 170.

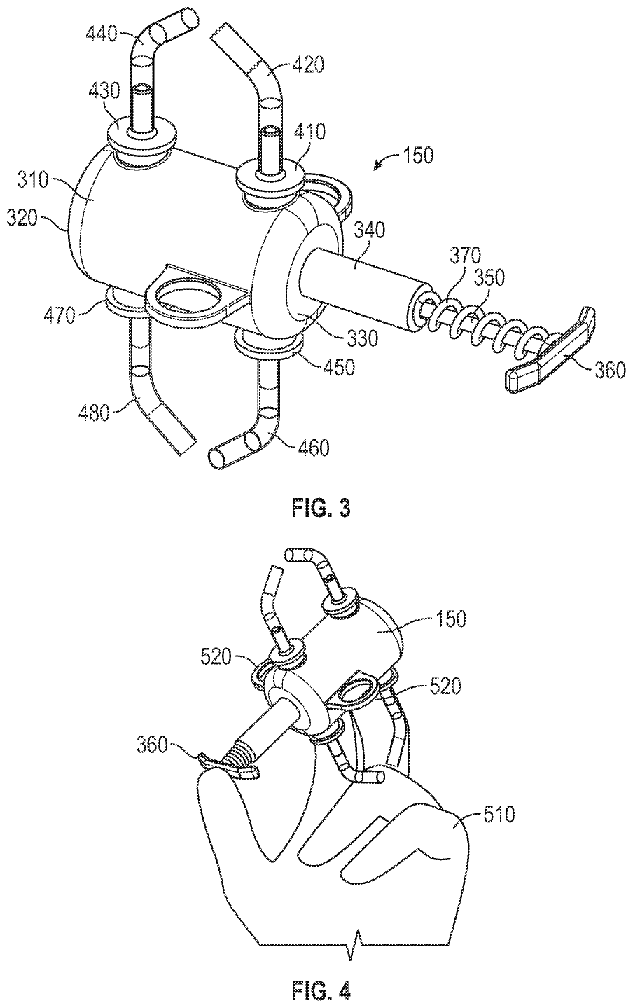

FIG. 3 illustrates a perspective view of a hand pump 150 in accordance with embodiments described herein. The hand pump 150 preferably includes a chamber 310 that may be generally cylindrical. The chamber 310 may have a forward chamber 320 and a rearward chamber 330. Extending in one direction from the chamber 310, for example, rearwardly from the rearward chamber 330 as illustrated in FIG. 3, is a piston slider 340. A push rod 350 is configured to extend into at least a portion of the piston slider 340 on one end and is connected to a grip handle 360 on an opposing end. A return spring 370 is preferably positioned between opposing surfaces on the grip handle 360 and the piston slider 340 to resist movement of the grip handle 360 toward the piston slider 340.

During use of the hand pump 150, the grip handle 360 is pressed forward toward the piston slider 340 or chamber 310. As the grip handle 360 is pressed forward toward the piston slider 340, the push rod 350 is advanced within the piston slider 340, and the return spring 370 is compressed between the advancing grip handle 360 and the piston slider 340. When the grip handle 360 is no longer pressed forward toward the piston slider 340 and the pressing force is removed from the grip handle 360, the return spring 370 expands to press the grip handle 360 away from the piston slider 340, thereby advancing the push rod 350 rearwardly out of the piston slider 340.

The operation of the hand pump 150 includes the reciprocating action of advancing the grip handle 360 and push rod 350 forward toward the chamber 310 to actuate the pump in one motion. The hand pump 150 then operates in a second motion when pressure against the grip handle 360 is released and the return spring 370 is allowed to expand and move the grip handle 360 and the push rod 350 rearwardly away from the chamber 310. As this action is repeated, the hand pump 150 pumps fluid toward the patient.

FIG. 3 further depicts a plurality of input and output ports for communicating fluid from the input line 130 to the output line 160 via the pump chamber 310. The hand pump 150 preferably includes a first input port 410 that is in fluid communication with a first input line 420 and the chamber 310 and is configured to conduct fluid from the first input line 420 into the chamber 310. A second input port 430 is in fluid communication with a second input line 440 and the chamber 310 and is configured to conduct fluid from the second input line 440 into the chamber 310.

The hand pump 150 preferably includes a first output port 450 that is in fluid communication with a first output line 460 and the chamber 310 and is configured to conduct fluid from the chamber 310 to the first output line 460. A second output port 470 is in fluid communication with a second output line 480 and the chamber 310 and is configured to conduct fluid from the chamber 310 to the second output line 480.

In operation, the reciprocating action of the hand pump 150 directs fluid through the ports to pump the fluid toward the patient. When the grip handle 360 is first advanced forward, fluid is expelled from the chamber 310 through the second outlet port 470, and during this first motion, fluid is drawn through the first input port 410 into the chamber 310. When the grip handle 360 is allowed to be drawn rearwardly by the return spring 370, the flow of fluid through the chamber 310 changes. During this second motion, fluid is expelled from the chamber 310 through the first output port 450 and is drawn into the chamber 310 through the second input port 430.

FIG. 4 illustrates operation of the hand pump 150 by a user's single hand 510. Single hand use of the pump 150 by providing finger grips 520 on the sides of the hand pump 150. In some instances, the operation of the hand pump 150 can by similar to that of a common syringe. With two fingers extending through the finger grips 520, a user can depress the grip handle 360 to actuate the pump 150 in a first direction. For the second direction of the pump's operation, the user can release pressure against the grip handle 360 while keeping the fingers through the finger grips 520. The fingers in the finger grips 520 will keep the pump 150 stabilized and in the user's hand, and releasing pressure against the grip handle 360 will allow the return spring 370 to advance the grip handle 360 rearwardly. This action can continue repeatedly to pump fluid to the patient by a single hand of the user.

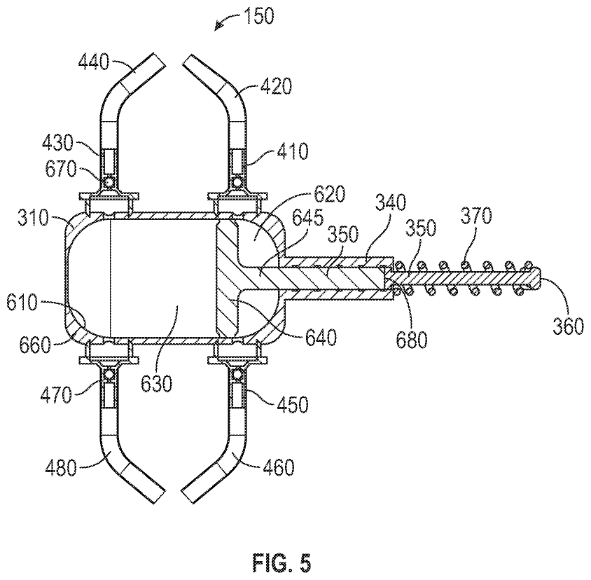

FIG. 5 illustrates a cross-sectional view of the dual chamber hand pump 150 that shows how the pump 150 can conduct fluid during each of the motions, or actions, of the pump 150 described herein. The chamber 310 of the hand pump 150 includes an internal chamber wall 610 that defines a first chamber portion 620 and a second chamber portion 630. The first chamber portion 620 and the second chamber portion 630 is separated by a piston disk 640 that is moveably mounted within the chamber 310. The piston disk 640 is preferably connected, either through assembly or integrally formed, with a piston rod 650 that extends rearwardly from the piston disk 640 and through at least a portion of the piston slider 340. In some embodiments, the piston disk 640 and piston rod 650 form a plunger. Although the disk is shown in embodiments described herein as a flat cylindrical disk, the disk can have other forms that are not cylindrical. As the piston disk 640 is advanced forwardly, toward a chamber forward end 660, the second chamber portion 630 decreases in volume, and the first chamber portion 620 increases in volume.

As described herein, fluid flow is provided to the chamber 310 from first and second input lines 420, 440 through first and second input ports 410, 430. Fluid flow is expelled from the chamber 310 to first and second output lines 460, 480 through first and second output ports 450, 470. The first input port 650 and the first output port 450 are in fluid communication with the first chamber portion 620. The second input port 430 and the second output port 470 are in fluid communication with eh second chamber portion 630. Each of the ports are shown with a valve 670 that controls fluid flow therethrough and to ensure the flow through the ports are one directional. These one-way valves ensure that while one of the chamber portions 620, 630 is expanding, fluid is drawn from the source into the chamber 310 and not from down line of the chamber 310. Likewise, these one-way valves ensure that while one of the chamber portions 620, 630 are contracting, fluid is expelled toward the patient 110 and not toward the fluid source 120.

Movement of the piston disk 640 within the chamber 310 is controlled by depressing or releasing the grip handle 360. When the grip handle 360 is pressed forwardly, the push rod 350 is advanced toward the chamber 310. A push rod forward end 680 coupled to or otherwise connected with the piston rod 650 advances the piston rod 650 through the piston slider 340 and moves the piston disk 640 towards the chamber forward end 660. When the grip handle 360 is released, the return spring 370 presses the grip handle 360 rearwardly and draws the piston disk 640 rearwardly away from the chamber forward end 660.

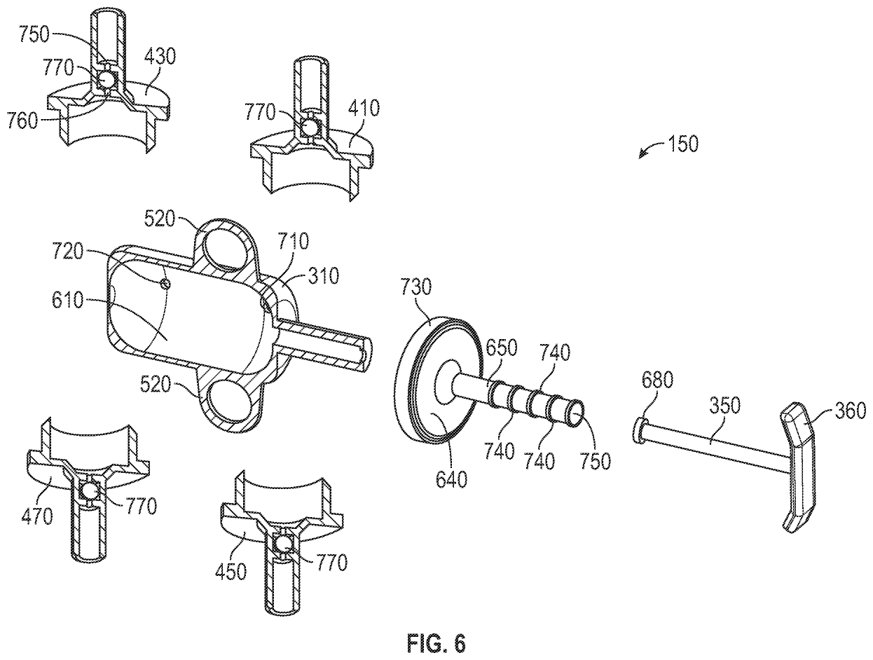

FIG. 6 illustrates a partial cross section exploded view of the hand pump 150. The chamber 310 is illustrated in a rotated cross-sectional view as compared to FIG. 5, depicting the finger grips 520 and a first inlet aperture 710 and a second inlet aperture 720 through which fluid is conducted into the chamber via the first input port 410 and the second input port 430, respectively. Although not depicted in FIG. 6, a first outlet aperture and a second outlet aperture are included in the chamber wall 610 separated from the first and second input ports 710, 720. As depicted in the figure, the chamber wall 610 defines a generally cylindrical chamber 310 that is configured to contain a piston disk 640 and reciprocally moves axially within the cylindrical chamber 310 along a general axis of the chamber 310.

The piston disk 640 preferably includes a piston ring 730 that circumferentially and/or peripherally extends around an outer edge of the disk 640 such that the piston ring 730 seals against the chamber wall 610 as the piston disk 640 moves within the chamber 310. The piston ring 730 seals against the chamber wall 610 to restrict fluid communication between the first chamber portion 620 and the second chamber portion 630. Extending from one face of the piston disk 640 is the piston rod 650, which includes one or more piston rod seals 740 axially spaced and separated along the piston rod 650. The piston rod seals 740 are configured to seal against an inner surface of the piston slider 340 to restrict fluid from being expelled from the chamber 310 through the piston slider 340. A piston rod rearward end 750 is configured to be coupled or connection to the push rod forward end 680 such that axial or rotational manipulation of the grip handle 360 will be transferred through the push rod 350, the piston rod 650, and to the piston disk 640.

The input ports 410, 430 and the output ports 450, 470 are each configured to connect to tubing at one part of the port and the hand pump 150 at another part of the port. The ports are configured to provide fluid communication between the tubing and the hand pump 150. The ports can include an inlet aperture 750 and an outlet aperture 760 that conduct fluid through each port. The ports preferably include a one-way valve, depicted in FIG. 6 as a ball valve 770, that permits fluid to pass through the respective port in only a single direction. For example, the first and second input ports 410, 430 may include a ball valve 770 that permits fluid flow from the first and second input lines 420, 440 into the chamber 310. They may also resist or prevent fluid flow from the chamber 310 toward the first and second input lines 420, 440. The one-way valve function may be accomplished by providing a ball valve seating profile for the ball that matches the contour of the ball on the side of the ball that leads to the first and second input lines 420, 440 while providing flow paths past or around the ball on the side of the ball that leads to the chamber 310. Accordingly, when fluid flows from the first and second input lines 420, 440, the ball is pressed toward the chamber 310 where flow paths past or around the ball are provided, thus permitting flow in that direction. When fluid pressure changes, such that flow would otherwise be conducted toward the first and second input lines 420, 440, the ball is pressed against the ball valve seating profile that matches the contour of the ball, and fluid is restricted or prevented from passing around the ball toward the first and second input lines 420, 440.

The first and second output ports 450, 470 operate in an opposite manner as that described with respect to the first and second input ports 410, 430. Each of the first and second output ports 450, 470 preferably includes a one-way valve, depicted in FIG. 6 as a ball valve 770, that permits fluid to pass through the respective port in only a single direction. The first and second output ports 450, 470 may permit flow from the chamber 310 toward the first and second output lines 460, 480 but resist or prevent fluid flow from the first and second output lines 460, 480 toward the chamber 310. The one-way valve function of ports 450, 470 may be accomplished by providing a ball valve seating profile for the ball that matches the contour of the ball on the side of the ball that leads toward the chamber 310 while providing flow paths past or around the ball on the side of the ball that leads to output lines 460, 480. Accordingly, when fluid flows from the chamber toward the output lines 460, 480, the ball is pressed toward the output lines 460, 480 where flow paths past or around the ball are provided, thus permitting flow in that direction. When fluid pressure changes, such that flow would otherwise be conducted toward the chamber 310, the ball is pressed against the ball valve seating profile that matches the contour of the ball, and fluid is restricted or prevented from passing around the ball toward the chamber 310.

While embodiments shown herein depict a one-way valve that illustrates a ball valve, other one-way valves may also be used in similarly coordinating manner to accomplish the same or similar functions as that described above. For example, duckbill valves, umbrella valves, flapper valves, and other one-way valves may be used in various embodiments to achieve the one-way function that is accomplished during the pumping process for the hand pump 150.

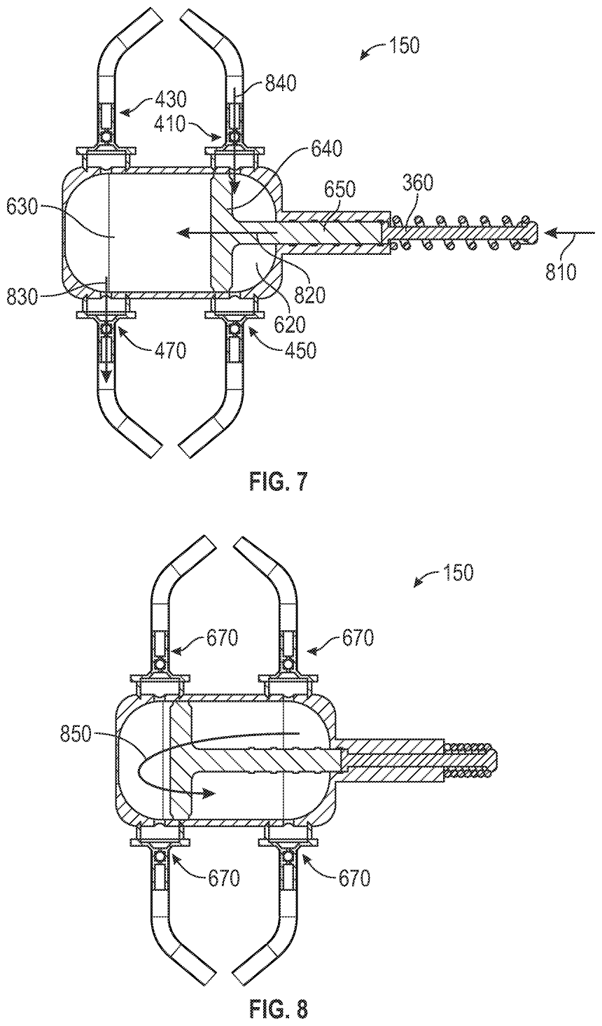

FIGS. 7-9 depict operation of the dual chamber hand pump 150. In order to operate the hand pump 150, either to infuse fluid to a patient or to prime a fluid line, the operator takes the hand pump 150 in hand and presses the grip handle 360 with a forward force 810. When the grip handle 360 is pressed with a sufficient forward force, the piston rod 650 and piston disk 640 will advance in the forward direction 820. Forward movement of the piston disk 640 will reduce the size of the second chamber portion 630, causing a positive pressure to be applied to the fluid in the second chamber portion 630. This positive pressure will cause fluid to be expelled from the chamber 310 through the second outlet port 470 as indicated by the arrow showing output flow 830. The second input port 430 preferably includes valve 670, which will prevent or restrict fluid from flowing through the second input port 430.

As the second chamber portion 630 decreases in size, the first chamber portion 620 increases in size, creating a negative pressure to form in the first chamber portion 620. An arrow depicting input flow 840 is illustrated, reflecting fluid flow through the first input port 410 toward the first chamber portion 620. This fills the first chamber portion 620 in balance with the fluid that is expelled from the second chamber portion 630. Fluid that may otherwise be drawn in through the first output port 450 is prevented or restricted by the valve 670 in the first output port 450.

FIG. 8 depicts when the grip handle 360 is fully depressed in the forward direction 820. At this point in the operation, substantially no fluid will be drawn through the input ports or the output ports, and the piston disk 640 experiences a reversing direction 850. The piston disk 640 stops moving forward and switches to a point ready to reverse its direction of movement.

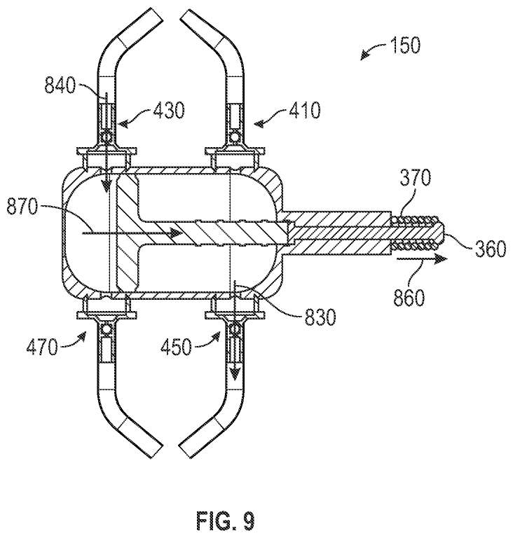

FIG. 9 illustrates an expanding force 860 applied by the compressed return spring 370, as the compressed spring is forced to expand against the grip handle 360. This force moves the piston rod 650 and the piston disk 640 in a rearward direction 870. As the piston disk 640 moves in the rearward direction 870, the first chamber portion 620 will reduce in size, creating a positive pressure in the first chamber portion 620. This positive pressure will cause output flow 830 from the chamber 310 through the first outlet port 450. Valve 670 in the first input port 410 prevents or restricts fluid from flowing through the first input port 410 from the first chamber portion 450.

As the first chamber portion 620 decreases in size, the second chamber portion 630 increases in size, creating a negative pressure to form in the second chamber portion 630. Input flow 840 is provided through the second input port 430 to fill the second chamber portion 630 in balance with the fluid that is expelled from the first chamber portion 620. Fluid is not drawn through the second output port 470 because valve 670 prevents or restricts fluid to flow through the second output port 470 toward the chamber 310.

The operation of reciprocal motion pressing the grip handle 360 and allowing it to expand under the force of the return spring 370 draws fluid into the chamber 310 and expels fluid from the chamber 310 with each stroke, thereby providing a relatively consistent flow operation for either infusing a patient or priming a fluid line. During operation, the hand pump 150 can pump fluid at a rate of 13-15 liters per hour, and in some embodiments, the hand pump 150 can pump fluid at a rate greater than 15 liters per hour.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, inserting and the like via a hardware element.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter. The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A single-hand pump for use with an intravenous delivery system, comprising:
a pump chamber comprising a plurality of ports that provide fluid pathways through a wall of the chamber housing; and
a plunger positioned within the chamber and configured to be reciprocally axially moveable in the chamber, the plunger separating the chamber into a first chamber and a second chamber;
wherein the first chamber is in fluid communication with a first plurality of ports, the second chamber is in fluid communication with a second plurality of ports, the first plurality of ports being fluidly isolated through the chamber from the second plurality of ports by the plunger,
wherein when the plunger is axially advanced toward the first plurality of ports, fluid is expelled from the chamber through a first of the first plurality of ports and restricted from being expelled through a second of the first plurality of ports, and fluid is conducted into the chamber through a first of the second plurality of ports and restricted from being conduct into the chamber through a second of the second plurality of ports, and
wherein when the plunger is axially advanced toward the second plurality of ports, fluid is expelled from the chamber through one of the second plurality of ports and restricted from being expelled through another of the second plurality of ports, and fluid is conducted into the chamber through a one of the first plurality of ports and restricted from being conduct into the chamber through another of the second plurality of ports.

2. The pump of claim 1, further comprising a return spring that is compressed when the plunger is advanced in a first direction and expands when the plunger is advanced in a second direction.

3. The pump of claim 1, further comprising a handle that is configured to be pressed by a palm of a user.

4. The pump of claim 3, further comprising a plurality of finger grips that are configured to be engaged by a user's fingers.

5. The pump of claim 4, wherein the single-hand pump is configured to be actuated by a single hand of a user as the plunger advances toward the first plurality of ports and as the plunger advances toward the second plurality of ports.

6. The pump of claim 1, wherein the first plurality of ports comprises at least two one-way valves.

7. The pump of claim 6, wherein the at least two one-way valves comprise ball valves.

8. The pump of claim 1, wherein the second plurality of ports comprises at least two one-way valves.

9. The pump of claim 8, wherein the at least two one-way valves comprise ball valves.

10. A single-hand pump for use with an intravenous delivery system, comprising:
a pump chamber comprising a first fluid pathway, a second fluid pathway, a third fluid pathway, and a fourth fluid pathway, the first and third pathways configured to conduct fluid to the chamber and to stop flow therethrough from the chamber, the second and fourth pathways configured to conduct fluid from the chamber and to stop flow therethrough to the chamber;
a piston positioned within the chamber, the piston being moveable between first and second positions and fluidly separating the first and second fluid pathways from the third and fourth fluid pathways within the chamber, wherein when the piston is moving toward the first position, fluid is conducted through the second and third fluid pathways and stopped through the first and fourth fluid pathways, and when the piston is moving toward the second position, fluid is conducted through the first and fourth fluid pathways and stopped through the second and third fluid pathways.

11. The pump of claim 10, further comprising first, second, third, and fourth ports fluidly coupled to the chamber providing respective flow along the first, second, third, and fourth fluid pathways.

12. The pump of claim 11, further comprising a plurality of one-way valves in each of the ports, the valves configured to conduct or stop the flow during movement of the piston.

13. The pump of claim 12, wherein the plurality of one-way valves comprises a ball valve.

14. The pump of claim 10, further comprising a plurality of finger grips that are configured to be engaged by a user's fingers.

15. The pump of claim 14, wherein the single-hand pump is configured to be actuated by a single hand of a user as the plunger moves between the first and second positions.

16. The pump of claim 10, further comprising a return spring that is compressed as the piston moves in one direction and expands as the piston moves in another direction.

17. The pump of claim 16, wherein the piston is configured to move in the one direction by a user's action, and wherein the piston is configured to move in the another direction by expansion of the return spring.

18. A method of conducting fluid through an intravenous delivery system using a single-hand pump, comprising:
providing a pump chamber comprising a first fluid pathway, a second fluid pathway, a third fluid pathway, and a fourth fluid pathway, the first and third pathways configured to conduct fluid to the chamber and to stop flow therethrough from the chamber, the second and fourth pathways configured to conduct fluid from the chamber and to stop flow therethrough to the chamber;
providing a piston positioned within the chamber, the piston fluidly separating the first and second fluid pathways from the third and fourth fluid pathways within the chamber;
moving the piston between first and second positions, and wherein when the piston is moving toward the first position, fluid is conducted through the second and third fluid pathways and stopped through the first and fourth fluid pathways, and when the piston is moving toward the second position, fluid is conducted through the first and fourth fluid pathways and stopped through the second and third fluid pathways.

19. The method of claim 18, further providing first, second, third, and fourth ports fluidly coupled to the chamber.

20. The pump of claim 19, further conducting and stopping fluid flow along the first, second, third, and fourth pathways via one-way valves.
